(19) United States
(12) Patent Application Publication
(10) Pub. No.: US 2015/0190543 A1
(43) Pub. Date: Jul. 9, 2015

(54) COFORM NANOFIBROUS SUPERABSORBENT MATERIALS

(71) Applicants: Larry Marshall, Chesterfield, VA (US); Michael Bryner, Midlothian, VA (US); Gary Huvard, Chesterfield, VA (US)

(72) Inventors: Larry Marshall, Chesterfield, VA (US); Michael Bryner, Midlothian, VA (US); Gary Huvard, Chesterfield, VA (US)

(73) Assignee: VERDEX TECHNOLOGIES INC., Richmond, VA (US)

(21) Appl. No.: 14/462,557

(22) Filed: Aug. 18, 2014

Related U.S. Application Data
(63) Continuation-in-part of application No. 14/148,712, filed on Jan. 6, 2014, now Pat. No. 8,808,594.

(51) Int. Cl. A61L 15/60; A61L 15/24; A61L 15/46; A61L 15/42; A61L 15/44

(52) U.S. Cl. CPC A61L 15/60; A61L 15/425; A61L 15/44; A61L 15/46; A61L 15/24; A61L 2300/252; A61L 2300/606

(57) ABSTRACT

A fibrous super absorbent material is disclosed including a) a hydrophilic three-dimensional fibrous web consisting of a first population of fibrillated nanofibers, and a second population of fibrillated microfibers, both populations uniformly distributed throughout the three-dimensional fibrous web where the first population comprises at least 50% of the total fiber population and b) a population of superabsorbent polymer (SAP) particles with a median size of less than 40 microns dispersed throughout the fibrous web. In various embodiments, a plurality of coarse (greater than 40 microns in diameter), fine from about (40 μm to about 10 μm in diameter), ultrafine (from about 10 μm to about one μm in diameter) and nanosize (less than one μm in diameter) particles are dispersed into the fibrous structure to absorb liquids or remove contaminants or bacteria from the fluids.

### PRIOR APPLICATION

This application is a continuation-in-part of U.S. patent application Ser. No. 14/148,712

### TECHNICAL FIELD

This application generally relates to superabsorbent nonwoven nanofibrous materials.

### DESCRIPTION OF THE RELATED ART

Highly absorbent or superabsorbent non-woven media are used in a variety of products including sanitary goods, hygienic goods, wiping cloths, water-retaining agents, dehydrating agents, sludge coagulants, disposable towels, thickening agents, condensation-preventing agents, wound care products and release control agents for various chemicals and pharmaceuticals. There is general understanding in industry that to be called superabsorbent, a material should imbibe, absorb or gel at least 10 times its own weight of fluid and retain it under moderate pressure. An important component of disposable absorbent articles such as diapers or wound care products is an absorbent core structure comprising super absorbent polymers, or SAPs, which ensure that large amounts of fluids, e.g. water, urine or blood can be absorbed by the article during its use.

SAPs are hydrocolloids capable of absorbing many times their own weight in liquids such as body exudates as a result of osmotic forces. SAPs are typically lightly crosslinked polymers or available in a variety of chemical forms, including substituted and unsubstituted natural and synthetic polymers, such as hydrolysis products of starch acrylonitrile graft polymers, carboxymethylcellulose, crosslinked polyacrylates, sulfonated polystyrenes, hydrolyzed polyacrylamides, polyvinyl alcohols, polyethylene oxides, polyvinylpyrrolidones, and polyacrylonitriles.

Incumbent superabsorbent materials are typically multi-layered, thick and heavy. Designers of absorbent articles have generally designed products responsive to consumer demands for less bulky, and lighter absorbent articles having a high absorption rate and high capacity. As a result, absorbent article designs have become progressively thinner, using various absorbent polymers with high absorbent capacity. For example, the thickness of a feminine hygiene pad has been reduced from about 15 mm to 20 mm in the mid 1980's to about 2.5 mm to 6 mm today. Most thin articles currently available are relatively rigid and less comfortable against the skin than prior thicker articles. There is therefore a need for thinner superabsorbent materials providing greater comfort and drapability.

Commercially, SAPs are widely used in personal hygiene products such as diapers and sanitary napkins. The SAP material is distributed typically on or in a matrix (i.e., a core) of natural or synthetic fibers. Because SAPs are highly cross-linked, it is difficult to put them into solution. Accordingly, SAPs are typically used in the form of powders, fibers, or granular particles (i.e., discrete units). When the SAPs were first introduced in absorbent articles, a significant decrease in the article's thickness was achieved, because a much smaller volume of super-absorbent polymer was needed, compared to the large volumes of absorbent pulp, traditionally used in absorbent articles.

Besides osmotic forces, absorbency based on capillary forces is also important in many absorbent articles such as a paper towel soaking up spilled liquids. Capillary absorbents can offer superior performance in terms of the rate of fluid acquisition and wicking, i.e. the ability to move aqueous fluid away from the point of initial contact. Absorbent articles such as diapers use a fibrous matrix as the primary capillary transport vehicle to move the initially acquired aqueous body fluid throughout the absorbent core so that it can be absorbed and retained by the SAP.

Although various materials based on fibrous superabsorbent cores have been suggested for use in absorbent articles, there is still a need for superabsorbent cores having optimized combinations of features and characteristics that would render such cores especially useful in commercially marketed absorbent products. In terms of desired absorbency characteristics, including capillary fluid transport capability, it has been determined that optimized absorbent, open-celled polymeric fibrous cores should have the following characteristics:

Polymeric fine fibers have found various commercial applications over the last three decades owing to their unique fiber size, which can broadly be defined as having fiber diameter of less than 5 microns. Polymeric fine fibers coupled with SAP particles considerably increases the absorbent capacity of the superabsorbent material with a significant reduction in material thickness.

Polymeric fine fibers can be made using a variety of different technologies; one such process is melt-blowing. Meltblowing technology utilizes a hot air flow to deform, accelerate and elongate a volume of melted polymer into a fibrous shape. Typically the melted polymer is extruded through a row of small tightly spaced spinneret holes. SAP particles can be injected into a separate air stream and mixed with the extruded fibers.

US Patent Application 20003/012915 teaches a process for forming a super absorbent composite for use in personal hygiene products, comprising a non-woven core with SAP uniformly distributed throughout the thickness of the core and bonded to the core with an adhesive. A non-woven core is provided to a processing line. An adhesive is introduced throughout the thickness of the core. Then the core is impregnated with a SAP by blowing a stream of SAP and air onto the core at a sufficiently high velocity to cause the super absorbent polymer to penetrate the surface of the core. The super absorbent polymer is distributed uniformly throughout the cross-section of the non-woven core and immobilized by the adhesive. Alternatively, the super absorbent polymer is blown into a non-woven core without adhesive and immobilized by a fiber matrix in the non-woven core.

U.S. Pat. No. 7,267,789 is directed to a method of forming nonwoven webs comprising particulates. The method of forming the nonwoven web generally comprising the steps of forming fibers from a melt fibrillation process, forming at least one fluid stream containing particulates, mixing said fibers with said particulates to form a fiber-particulate mix, and depositing the mix on a surface to form a web. The nonwoven web will have the particulates entrapped in the web. The nonwoven web may comprise a layer having a significant number of nanofibers with diameters less than one micron.

U.S. Pat. No. 8,487,156 is directed to hygiene articles comprising nanofibers. The nanofiber webs can be used as a barrier, wipe, absorbent material, and other uses. Particularly, the nanofiber web is used in a diaper as a barrier-on-core, outercover, and/or leg cuff. It may also be used as a wipe for reducing the gradient of liquid, controlled delivery of materials, and other uses. The nanofibers, having a diameter of less than 1 micron, must comprise a significant number of the fibers in one layer of the web contained by the hygiene article. The nanofibers are produced from a melt film fibrillation process. The process includes the steps of providing a polymeric melt, utilizing a central fluid stream to form an elongated hollow polymeric film tube, and using this and/or other fluid streams to form multiple nanofibers from the hollow tube.

Coform nonwoven webs or coform materials are known in the art and have been used in a wide variety of applications, including superabsorbent. The term “coform material” means a composite material containing a mixture or stabilized matrix of thermoplastic filaments and at least one additional material, often called the “secondary material”. Examples of the secondary material include, for example, absorbent fibrous organic materials such as woody and non-wood pulp from, for example, cotton, rayon, recycled paper, pulp fluff; superabsorbent materials such as superabsorbent particles and fibers; inorganic absorbent materials and treated polymeric staple fibers, and other materials such as non-absorbent staple fibers and non-absorbent particles and the like. Exemplary coform materials are disclosed in commonly assigned U.S. patent application Ser. No. 14/148,712 (Marshall et al.).

Current coform superabsorbent materials generally comprise large size SAP particles which tend to block the pores of the materials when wetted. U.S. Pat. No. 5,061,259 (Goldman et al.) teaches absorbent structures comprising hydrophilic fiber material and nonfragile particles of polymeric gelling agent. The gelling agent particles incorporated into such structures are selected to have a mass median particle size ranging from about 400 to 700 microns. Such large particles require thick absorbent webs. In absorbent materials, both the total absorbent capacity and the rate of absorbence are important. While Goldman, et al, found a novel way of maximizing absorbent capacity by incorporating a specific range of large SAP particles, the large particles necessarily limit the absorption rate. The absorption rate depends upon the rate at which liquid can penetrate the SAP particles and this depends upon the surface area of the particles. For example, a 40 micron SAP particle will have 100× the surface per unit mass compared to a 400 micron particle. Thus, to maximize the absorption rate of an absorbent material, it is desirable to incorporate much smaller SAP particles. There is therefore a need for coform superabsorbent materials able to hold SAP with a fine particle size which can maintain a high absorbency rate after being wetted.

Thermoplastic fibers, such as polyesters and polyolefins are generally used in superabsorbent materials for economic, aesthetic and strength reasons. However, polypropylene is, by its nature, hydrophobic. When spun into fibers or filaments which are used to form a fabric, the resulting fabric is also hydrophobic or non-wettable. This results in delay of liquid absorption at the surface of the fabric. Thus, the fabric must be specially treated or altered in some way to render the fabric wettable, that is, able to allow the passage or transfer of fluids, if the fabric is to be suitable for use as an inner lining fabric for a sanitary article. It has been known in the art to impart wettability to a polyolefin fabric by adding a surfactant to the polymer melt as in U.S. Pat. No. 8,663,517. This has the disadvantage of changing the viscosity and physical characteristics of fibers made from the polymer melt. Other approaches such as dipping the fabric in a water solution containing a surfactant have shown moderate improvements in wettability as the surfactant will tend to clog the pores of the fabric. There is therefore a need for superabsorbent materials with small pores and a high degree of wettability.

The absorbent properties of SAPs are attributed to the electrostatic repulsion between the charges along the polymer chains, and the osmotic pressure of the counter ions. It is known that these absorption properties are drastically reduced in solutions containing electrolytes, such as saline, urine, and blood. The polymers function much less effectively in the presence of such physiologic fluids. The decreased absorbency of electrolyte-containing liquids is illustrated by the absorption properties of a typical, commercially available SAP, i.e., sodium polyacrylate, in deionized water and in 0.9% by weight sodium chloride (NaCl) solution. The sodium polyacrylate can absorb 146.2 grams (g) of deionized water per gram of SAP (g/g) at 0 psi, 103.8 g of deionized water per gram of polymer at 0.28 psi, and 34.3 g of deionized water per gram of polymer of 0.7 psi. In contrast, the same sodium polyacrylate is capable of absorbing only 43.5 g, 29.7 g, and 24.8 g of 0.9% aqueous NaCl at 0 psi, 0.28 psi, and 0.7 psi, respectively. The absorption capacity of SAPs for body fluids, such as urine or blood, therefore, is dramatically lower than for deionized water because such fluids contain electrolytes. This dramatic decrease in absorption is termed “salt poisoning.” There is therefore a need for superabsorbent materials which remain effective in the presence of solutions containing electrolytes.

In recent years product designers have shifted their design focus to addressing aesthetic and skin-wellness issues, including the removal of unpleasant odors. The odor absorption methods includes incorporation into the absorbent article of compounds that are known to absorb odors, such as activated carbons, clays, zeolites, silicates, cyclodextrine, ion exchange resins and various mixture thereof. Some of the odor absorbing particles lose odor-trapping efficiency when they become moist, as most absorbent articles do. Furthermore, in order for these reagents to be effective at controlling odor, a high loading of these reagents is required which increases the cost and weight of the absorbent article, and tends to adversely affect the absorbency and performance of the absorbent article. There is therefore a need for superabsorbent materials which incorporate highly effective odor absorbing particles.

Anti-microbial agents have also been applied to the surface of the fabric, although such are very limited in preventing bacterial growth, since the anti-microbial agent is located outside the body fluid accumulation zone—i.e., the absorbent core of the absorbent article is There is therefore a need for superabsorbent materials with anti-bacterial agents dispersed throughout the superabsorbent core.

### SUMMARY

The present specification discloses new hydrophilic non-woven fibrous media for liquids that satisfies the need for thin, high absorbent capacity materials for use in hygiene and wound care articles.

It is an object of an embodiment of the disclosure to provide a superabsorbent media that provides a higher absorbent capacity and absorbency rate per unit of weight than conventional superabsorbent materials. It is still a further object in an example of an embodiment of the disclosure to provide media that has a saline solution absorbent capacity greater than about 30 times its dry weight and has a rate of absorbency that is greater than about 20 ml of saline solution per gram of material per second.

Accordingly, it is an object of the present disclosure to provide a superabsorbent material in the form of a very thin, light and easy to manufacture product, more comfortable and drapable than similar products of the prior art.

In an embodiment, the present disclosure satisfies the need for a non-woven fibrous medium that retains ultrafine particles of less than 40 microns in diameter or nanoparticles without the need for binders or adhesives.

It is another object in an embodiment of the present disclosure to provide a superabsorbent material with a hydrophilic fibrous matrix.

It is another object of the disclosure to provide a superabsorbent material which is highly effective in the presence of fluids containing electrolytes.

It is a further object in an example of an embodiment of the present invention to produce a superabsorbent medium that has a porosity greater than 75% and is lighter and thinner than conventional super absorbent materials.

It is still a further object in an example of an embodiment of the disclosure to provide a superabsorbent material that has a mean pore size smaller than about 40 microns.

It is yet another object in an example of an embodiment of the present disclosure to provide a superabsorbent material that offers higher loft and a greater resistance to compression than conventional materials.

It is still a further object in an example of an embodiment of the present disclosure to provide a superabsorbent material that filters odors and contaminants and that has minimal impact on material thickness and weight.

It is yet another object in an example of an embodiment of the present disclosure to provide a superabsorbent material that includes a superabsorbent core as well as a hydrophilic contact layer.

It is yet another object in an example of an embodiment of the present disclosure to provide a superabsorbent material that includes a superabsorbent core as well as a hydrophobic backing layer.

It is another object in an example of an embodiment of the present disclosure to provide a hydrophilic coating of the super absorbent material that improves wettability of the material.

It is also an object in an example of an embodiment of the present disclosure to incorporate a nonionic surfactant homogeneously dispersed throughout the superabsorbent material to increase the absorbency rate of the material.

It is yet another object in an example of an embodiment of the disclosure to provide a media that filters odors and contaminants.

It is also object in an embodiment of the present disclosure to provide a non-woven medium that removes soluble and volatile organics from fluid streams.

It is also an object in an embodiment of the present disclosure to impart antibacterial properties to the super absorbent materials by incorporating powdered, metal oxide nanoparticles into a non-woven scaffold.

It is still a further object in an embodiment of the present disclosure to provide a non-woven material containing fine or nanosize powder that is held to the material to minimize dusting.

It is still a further object in an embodiment of the present disclosure to incorporate finely powdered therapeutic agents selected from the list consisting of nanosize iodine delivery agents, nanosize metal ion delivery agents, nitrous oxide delivery agents, nanosize polymer capsules for controlled drug delivery into a non-woven medium.

It is still a further object to in an embodiment of the present disclosure to impart germicidal properties to the fibrous material by solubilizing iodine in a nonionic surfactant to form a surfactant-iodine complex coating the fibrous matrix.

It is still a further object in an embodiment of the present invention to incorporate biologically active components such as growth factors, DNA or RNA into a non-woven medium.

More generally, the present disclosure is directed at fibrous super absorbent materials including a) a hydrophilic three-dimensional fibrous web consisting of a first population of fibrillated nanofibers, and a second population of fibrillated microfibers, both populations uniformly distributed throughout the three-dimensional fibrous web where the first population comprises at least 50% of the total fiber population and b) a population of superabsorbent polymer (SAP) particles with a median size of less than 40 microns dispersed throughout the fibrous web.

In an embodiment, the second population is comprised of fine fibers whose average diameter is larger than the average diameter of the nanofibers of the first population by a factor of at least 5. The second population of fine fibers is mixed with the nanofibers in order to provide a lofty and pressure resistant scaffold. In examples, the mean pore size from the fine fibers is less than about 40 microns. In various embodiments, a plurality of coarse (greater than 44 microns in diameter), fine from about (44 μm to about 10 μm in diameter), ultrafine (from about 10 μm to about one μm in diameter) and nanosize (less than one μm in diameter) particles are dispersed into the fibrous structure to absorb liquids or remove of contaminants or bacteria from the fluids. In an embodiment of the disclosure the density of the superabsorbent material is less than 0.25 grams per cubic cm.

In existing art, conventional superabsorbent media is produced with large fibers and pore sizes and, therefore the average size of particles dispersed and held therein is relatively large. This is turn results in reduced particulate surface area per unit of weight. To maximize the liquid absorption rate, a larger particulate surface area per unit weight is required, which in turn requires smaller particles and means to hold the smaller particles within the media. Therefore, in another embodiment, the fibrous material comprises a population of nanofibers homogeneously dispersed throughout the fine fiber matrix. The inclusion of nanofibers provides smaller pore sizes so that fine and nanosize particles can be loaded into or onto the media. In examples, fine particles with a median diameter smaller than 40 microns are dispersed within the fiber matrix. While not wishing to be bound by theory, ultrafine and nanosize particles that have diameters that are smaller than the average pore size of the fibrous structure are retained by thermal bonding on the nanofibers. Particles larger than the pore size of the media are held largely by mechanical entrainment. No binders are used in the fibrous structure that would envelop or otherwise reduce the effectiveness of the particles dispersed in the fibrous matrix.

The present disclosure is aimed at hydrophilic superabsorbent non-woven coform materials which can rapidly uptake fluids (high absorbency rate) and hold large amounts of liquids under pressure (high absorbent capacity). In an embodiment of the disclosure, the superabsorbent coform material comprises a hydrophilic three-dimensional fibrous web comprising a first population of fibrillated nanofibers and a second population of fibrillated microfibers, both populations uniformly distributed throughout the three-dimensional fibrous web, and a population of superabsorbent polymer (SAP) particles with a mean size of less than 40 microns dispersed throughout the fibrous web, where the liquid absorbent capacity of the superabsorbent material is greater than 30 times its dry weight.

A unique melt-film fibrillation process such as the one described in copending application U.S. Ser. No. 14/148,712 can produce an absorbent nanofibrous structure with a high rate of liquid uptake and which can maintain the high rate of uptake even after the absorbent structure has been previously wetted with one or more liquid insults.

Furthermore, such a process can also produce an absorbent structure with homogeneously distributed SAP with a fine particle size, with improved user comfort and rewetting performance.

In an embodiment of the current disclosure, a nonionic surfactant is injected into a heated pressurized gas stream before supplying the stream to a spinning nozzle where it is atomized and mixed with the polymer. The surfactant is thereby uniformly distributed on the surface of the polymeric fibers.

In another embodiment, the present disclosure is directed to a multi-layered superabsorbent material comprising a thin fibrous hydrophilic contact layer, a lofty hydrophilic non-woven distribution layer containing hydrophilic fibers to rapidly absorb the liquid from the contact layer, a superabsorbent core to store liquids absorbed through the distribution layer containing hydrophilic fibers and SAP particles and a hydrophobic, liquid impermeable and vapor permeable backing layer.

In another embodiment, the liquid-impermeable backing layer has a porosity engineered to achieve varying moisture vapor transmission rates (MVTR) according to the amount of liquid absorbed.

Numerous modifications and variations of the present invention are possible in light of the above teachings. It is therefore to be understood that within the scope of the appended claims, the invention may be practiced otherwise than as specifically described herein.

### BRIEF DESCRIPTION OF THE DRAWINGS

The invention, together with further objects and advantages thereof, may best be understood by reference to the following description taken in connection with the accompanying drawings in which:

FIG. 1 is an illustration of the SAP particle and fiber entanglement in a material of the disclosure.

FIG. 2 is an illustration an embodiment of the coforming process.

FIG. 3 is a chart of the fiber size distribution in an embodiment.

FIG. 4 is a chart of the fiber size distribution in another embodiment

FIG. 5 is an SEM of an embodiment of the disclosure.

FIG. 6 is an SEM of another embodiment of the disclosure.

FIG. 7 is a chart of the fiber size distribution in yet another embodiment

### DEFINITIONS

As used herein, the term “coform nonwoven web” or “coform material” means composite materials comprising a three-dimensional matrix of thermoplastic filaments and at least one additional material, usually called the “second material”. As an example, coform materials may be made by a process such as disclosed in patent application Ser. No. 14/148,712 in which at least one nozzle die head is arranged near a chute through which the second material is added to the web while it is forming. The second material may be, for example, superabsorbent particles and fibers; inorganic absorbent materials and treated polymeric staple fibers and the like; or a non-absorbent material, such as non-absorbent staple fibers or non-absorbent particles such as activated carbon. Exemplary coform materials are disclosed in commonly assigned U.S. patent application Ser. No. 14/148,712 the entire content of which is hereby incorporated by reference.

As used herein, the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Meltblown fibers, which may be continuous or discontinuous, and are generally smaller than 10 microns in average diameter.

As used herein, the phrase “nanofibers” refers to filaments having an average fiber diameter less than about 1 μm.

As used herein, the phrase “fine fibers” is intended to represent filaments having an average fiber diameter from about 10 microns to about 1 μm.

As used herein, a “coarse particle” is defined to be a particle having average particle diameter size that is greater than about 325 mesh (44 μm).

As used herein, a “fine particle” is defined to be a particle having average particle diameter size that is between about 44 μm and about 10 μm.

As used herein, an “ultrafine particle” is defined to be a particle having an average particle diameter size that is between about 1 μm and about 10 μm.

As used herein, the phrase “nanoparticles” refers to particles having an average diameter less than about 1 μm.

As used herein, the terms “hydrophilic” and “wettable” are used interchangeably to refer to a material having a contact angle of water in air of less than 90 degrees. The term “hydrophobic” refers to a material having a contact angle of water in air of at least 90 degrees.

As used herein the term “superabsorbent” refers to a material, natural or synthetic, capable of absorbing at least about 10 times its weight in liquid.

As used herein “absorbent capacity” of an absorbent material measured as the volume of liquid absorbed per unit weight of the absorbent material of a sample of the material cut into a square of length and width=2.54 cm and soaked in a 0.9% saline solution for 3 minutes and allowed to drain for 1 minute.

As used herein “absorbency rate” of a nonwoven material refers to the inverse of the strikethrough time, which is the time taken for a known volume of saline solution applied to the surface of a test piece of the nonwoven material, which is in contact with an underlying standard absorbent pad, to pass through the material. The liquid strikethrough time is measured according to ISO 9073-8 Textile Testing Standard.

### DETAILED DESCRIPTION

The disclosure relates to coform nanofibrous superabsorbent materials that can provide high absorbent capacity and a high absorbency rate.

### The Fiber Matrix

Absorbency characteristics are important determinants of the effectiveness of absorbent materials and the effectiveness of treatments to modify the surface characteristics of these materials. These characteristics are a function of both the micro and macrostructure of the absorbent material such as the capillary structure of the material, the pore size of the material, the chemical structure of the polymeric fibers, the structure of the surface of the absorbent material which contacts the liquid, the chemical and physical treatment of the absorbent and the multiple plied structure of complex absorbent materials.

The absorbent capacity is mainly determined by the interstitial space between the fibers (porosity), the absorbing and swelling characteristics of the material and the resiliency of the web in the wet state. In a non-woven fibrous structure, porosity is inversely correlated with fiber size. Smaller fibers will produce lighter structures of greater porosity and absorbent capacity. On the other hand, smaller fibers result in less resilient structures reducing the structure's ability to absorb liquids under pressure. It is therefore desirable to have a certain amount of larger fibers throughout the fiber media as it provides a scaffold against which higher pressure can be applied without collapsing the fibrous web. The resistance to pressure is dependent on the percentage of larger fibers contained in the fibrous web. If the percentage is too low the scaffold will collapse and the loftiness of the structure can no longer be maintained. This is turn will reduce the absorbency rate as porosity decreases together with the closing of pores. On the other hand, if the percentage of large fibers becomes too large then capillarity will remain low. Wettability is a function of pore size and larger pores will let more particles through. An optimally wettable structure is therefore a structure which can maintain its pore size after multiple insults. Coarse SAP particles, typically in the range from about 150 to 800 microns, will tend to block the pores of the fibrous structures when wetted. In an embodiment of the disclosure, fine SAP articles with an average size of less than about 50 microns is used, resulting in greater permeability to liquids and improved rewetting or strikethrough performance.

| | | BAS. | THICK- | ABS. | ABS. |
|---|---|---|---|---|---|
| | | WEIGHT | NESS | CAP. | RATE |
| EXAMPLE | SAP MATERIAL | (g/m2) | (mm) | (ml/g) | (ml/g/s) |
| Pampers | SPA | 669 | 1.3 | 18 | 3 |
| Ex. 2 | REON | 250 | 1.3 | 31 | 9 |
| Ex. 3 | AP-75 | 272 | 2.2 | 19 | 6 |
| Ex. 4 | Polyacrylamide | 234 | 3.3 | 19 | 13 |
| Ex. 5 | SPA | 175 | 1.6 | 32 | 23 |

Nanofibers are important to the performance of the absorbent material, especially the rate of absorbency. In considering why the rate of absorbency is improved with smaller fiber size, it is helpful to refer to the concept of capillary pressure. This is the pressure that allows a liquid to spontaneously penetrate a porous medium such as a fiber network. The capillary pressure is quantified (for a model cylindrical capillary) by an expression known as the Laplace equation:

ΔP=(2σ cos θ)/r where

ΔP=capillary pressure, σ=liquid surface tension, θ=contact angle, r=effective pore radius. All else being equal, the smaller the pore size the higher the capillary pressure, the higher the force impelling a liquid to enter and remain in the porous network. From this equation, we can see that by decreasing the pore radius r, the capillary pressure will increase. As more nanofibers are incorporated in a fibrous structure, the effective pore radius is decreased, which increases the capillary pressure. This results in higher absorbency rates up to the point at which other factors, such as liquid holding capacity, begin to have an effect.

It is known in the art that that mean pore size is a function of density and fiber size. The Bryner Model (Jrn1 of Eng. Fibers and Fabrics, Vol 2, Is. 1-2007) results for nonwoven fabrics show that the mean pore size, D_{p,mean} is directly proportional to fiber size and inversely proportional to the fiber volume fraction.

D_{p,mean}=πD_{f}/8(1−ε) where

D_{p,mean}=mean pore diameter, D_{f}=fiber diameter, ε=porosity.
In an example of an embodiment of the disclosure a superabsorbent material of the disclosure made from a polypropylene polymer has a basis weight of 60 gsm, average fiber size of 1.9 microns and a thickness of 1.6 mm for a density of 37.5 gsm per mm or 0.0375 g/cm³. With the PP density of 0.946 g/cm³ we arrive at a porosity or void fraction equal to (1-0.0375/0.946) or 96%. Under the above formula we find D_{p,mean}=πD_{f}/8*0.04=10×D_{f}
For fibrous webs in examples of embodiments of the current disclosure, the average pore size is about 10 times the average fiber size. With an average fiber size of 1.9 microns in the above embodiment, we arrive at a mean pore size of about 19 microns. In another embodiment, we find an average fiber size of 0.76 microns and a mean pore size of 7.6 microns. Therefore, absorbent materials with large numbers of nanofibers will result in highly porous structures with smaller pore sizes than materials made from larger microfibers only. This has the advantage of allowing the fibrous structure to hold large amounts of fine SAP particles without the use of binders. It is a specific aspect of the present disclosure to disclose superabsorbent materials comprising a substantial amount of nanofibers and fine fibers resulting in fibrous webs with high porosity and small pore structure entangling smaller SAP particles than materials of the prior art. Smaller SAP particles in turn result in greater surface area per unit of weight and therefore greater absorbency rate per unit of weight than larger SAP particles.

Superabsorbent materials of the disclosure support SAP loading rate greater than 60% of the total weight of the superabsorbent materials. Absorbent materials of the prior art, only have particle loading rates of 20 to about 50%. Superabsorbent materials of the disclosure provide structures with SAP particle loading rates as high as about 80%. This is in large part due to the high porosity of the materials of the disclosure. In an example of an embodiment of the disclosure, a coform superabsorbent material with a basis weight of 175 gsm and a thickness of 1.6 mm comprising a PP fibrous structure with a basis weight of about 60 gsm and density of 0.946 g/cm3 and a SAP basis weight of about 115 gsm and density of 1.22 g/cm3. The void fraction of the coform structure is 1−(0.0060/0.16/0.946+0.0.0115/0.16/1/22)=1−(0.039+0.059)=90.1%. The porosity of the fibrous matrix of the above embodiment of a material of the disclosure is greater than 96% without the SAP, while the porosity of the superabsorbent material with the dispersed SAP inside the fiber matrix is greater than 90%. The SAP particle loading rate of greater than 65%. When calendered to an ultrathin 0.7 mm, the porosity is still greater than about 77%. The highly porous and lofty materials of the disclosure can therefore easily expand when wetted without any detrimental effects to the integrity of the fibrous structure holding the SAP particles in place. The SAP-containing fiber matrix for use in the superabsorbent materials of the disclosure are sufficiently open for hydrogel that is formed when aqueous liquids are absorbed by the SAP particles to not completely fill the available interstitial volume of the material, and inhibit the rate of fluid uptake past the swollen SAP particles into the rest of the superabsorbent material.

Polymer nanofibers are known, however their use in absorbent materials has been very limited due to their fragility to mechanical stresses, limited porosity and the susceptibility of nanofiber webs to fuse under applied pressure. The coform fibrous materials described in this invention address these limitations by combining a population of nanofibers with a population of fine fibers: the smaller fibers provide smaller pore size and increased capillary pressure and rates of absorbency while the large fine fibers provide loft and increased resistance to pressure.

Preferably, a significant portion of the fibers should have an average diameter less than about 1000 nanometers. When the absorbent material is produced by a melt-film fibrillation process from polymeric nanofibers, such fibrous webs also have high porosity and high loft. Fibrillated fibers of the disclosure combine exceptionally fine dimensions and three-dimensional structure. Preferably, the nonwoven fibrous web comprises a first population of fibrillated polymeric nanofibers having an average diameter of less than about 1,000 nanometers and a second population of fibrillated polymeric fine fibers having an average diameter of less than about 5 microns, with the first population comprising more than 50% of total fiber population.

Typically, a layer of SAP particles is bonded to the surface of a fibrous absorbent layer, such as for example an air laid nonwoven. In the superabsorbent material of the disclosure, the SAP particles are homogeneously distributed throughout a three dimensional fibrous web. In an embodiment of the process for producing the coform superabsorbent materials, the process comprises supplying a first phase comprising a polymer melt and a second phase comprising a heated pressurized gas/surfactant stream to a two-phase flow nozzle, providing a second separate stream containing SAP particles and any additional particles where the second stream is naturally aspirated into the two-phase flow nozzle, commingling the first stream and second stream to form a composite stream and depositing the composite stream onto a receiving surface as a three dimensional web wherein the SAP particles are homogeneously distributed throughout the three dimensional web and held in place in the fibrous web without adhesives or binders.

Thermoplastic polymers materials that can be used in the compositions of the embodiments of the disclosure include materials such as polyolefins and mixtures thereof, polyacetals, polyamides, polyesters, polyalkylene sulfide, polyarylene oxide, polysulfones, modified polysulfone polymers and mixtures thereof, soluble polymers including polyacrylamide, polyacrylates, acrylamide-dimethylaminoethyl acrylate copolymers, polyamines, polyethyleneimines, polyamidoamines, polyethylene oxide. In examples of an embodiment of the disclosed materials, the fibers of the first stream are formed from a fiber forming material comprising a polymer melt or solution selected from polypropylene (PP), polyethylene including high density polyethylene (HDPE), medium density polyethylene (MDPE), low density polyethylene (LDPE), linear low density polyethylene (LLDPE), and/or very low density polyethylene (VLDPE), polyethylene terephthalate (PET), polybutylene succinate (PBS), polybutylene terephthalate (PBT), polystyrene (PS), polyacrylonitrile (PAN), polycarbonate (PC), polyvinyl chloride (PVC), polyvinylidene fluoride (PVDF), polyvinylidene chloride (PVDC), polytetrafluoroethylene (PTFE), polyphenylene sulfide (PPS), polyethersulfone (PES) and polysulfone (PSU), polymethyl methacrylate (PPMA), polyurethane (PUR), polyamide (PA), aliphatic polyesters including polylactic acid (PLA), polyglycolic acid (PGA), polylactic-co-glycolic acid (PLGA) and polycaprolactone (PCL). Furthermore, the fibrillated fibers may be produced in large quantities using equipment of modest capital cost. It will be understood that polymers other than those listed above may be fibrillated to produce extremely fine fibers. A thermoplastic polymer may be functionalized to have additional benefits such as absorbency, wettability, conductivity, antimicrobial properties, biodegradability, water solubility or dispersability.

### Particles

Copending application Ser. No. 14/148,712 teaches various examples of particles dispersed within a fibrous web of fibrillated nanofibers. SAP particles are particular examples of particles which can be added to impart greater absorbency rates and capacity to non-woven materials. Other particles can be added to impart odor-control, an electrostatic charge, anti-bacterial or therapeutic properties to the non-woven material.

Superabsorbent polymers (SAPs) are materials that have the ability to absorb and retain large volumes of water and aqueous solutions. This makes them ideal for use in water absorbing applications such as diapers and adults incontinence pads to absorbent medical dressings and controlled release medium. Superabsorbent materials of the prior art generally include relatively large SAP particles. U.S. Pat. No. 7,935,860 teaches SAP composition particles generally including particle sizes ranging from about 50 to about 1000 microns, such as from about 150 to about 850 microns. U.S. Pat. No. 6,159,591 teaches that to get the best results, SAP particles need to have a size be from about 150 to about 800 μm.

Smaller particles are generally disfavored because of the tendency of the SAP particles to agglomerate and inhibit the overall fluid permeability of the material a problem referred to as “gel blocking”. It has unexpectedly been found by the inventors that a fibrous network comprising fine fibers and nanofibers entangling the SAP particles, can actually overcome gel blocking, keeping the SAP particles from agglomerating, and result in greater absorbency rates without a loss in absorbent capacity. In an embodiment of the disclosure, illustrated in FIG. 1, as the SAP particles expand, the fibrous web expands as well keeping the SAP articles separate and preventing gel blocking from occurring.

Early SAPs were made from chemically modified starch and cellulose and other polymers like poly(vinyl alcohol) PVA, poly(ethylene oxide) PEO all of which are hydrophilic and have a high affinity for water. When lightly cross-linked, chemically or physically, these polymers become water-swellable but not water-soluble. SAPs with high absorbency under load (AUL) are made from partially neutralised, lightly cross-linked poly(acrylic acid), which has been proven to give the best performance versus cost ratio. The polymers are manufactured at low solids levels for both quality and economic reasons, and are dried and milled in to granular white solids. In water they swell to a rubbery gel that in some cases can be up to 99 wt % water. In an example of an embodiment of the present disclosure, the superabsorbent material comprises SAP particles consisting of a hydrocolloid, preferably an ionic hydrocolloid. Exemplary of superabsorbent material suitable for use in the present invention are cross-linked polyacrylates including sodium polyacrylate and polyacrylamides; cross-linked copolymers of maleic anhydride; polyvinyl alcohol; polyvinyl ethers; hydroxypropyl-cellulose (HPC); carboxymethyl-cellulose (CMC); carboxymethyl starch (CMS); polymers and copolymers of vinyl sulfonic acid; graft copolymers on polysaccharides such as chitin, chitosan, cellulose, starch, natural gums and polypeptide-based copolymers such as saponified starch-graft polyacrylonitrile copolymer made from corn (Reon).

In an embodiment of the disclosure, the mean diameter of the SAP particles is less than about 40 microns. Smaller particles offer greater surface area per unit of weight and therefore greater rates of absorbency.

### Odor-Control Agents

In another embodiment, the fibrous web includes odor controlling agents selected from the group consisting of clays, silicas, zeolites and molecular sieves. In still another embodiment, urease inhibitors are used to prevent production of ammonia including metal ions selected from the group consisting of silver, copper, iron, nickel, manganese, cadmium, cobalt, lead or palladium. Organic compounds known as urease inhibitors include N-(n-butyl) thiophosphoric triamide, N-(n-butyl)phosphoric triamide, thiophoshoryl triamide, phenyl phosphorodiamidate, cyclohexyl thiophosphoric triamide, cyclohexyl phosphoric triamide, phosphoric triamide, hydroquinone, P-benzoquinone, hexaamidocyclotriphosphazene, thiophyridines, thiophyrimidines, thiophyridine-Noxides, NN-dihalo-2-imidazolidinone, N-halo-2-oxazolidinone. The odor controlling agents can be added to the second separate stream as fine particles or atomized fluid or in a controlled manner and homogeneously distributed throughout the three-dimensional fibrous web.

### Therapeutic Agents

In still another embodiment of the process, the fibrous web includes therapeutic materials homogeneously distributed throughout the three-dimensional web. Therapeutic materials may include antimicrobial agents comprising metal ion nanoparticles selected from the list consisting of gold, silver, titanium, copper, cobalt, manganese, platinum, palladium, tin, bismuth, lead and zinc, drug loaded polymeric nanocapsules, iodine delivery agents selected from the list consisting of starch-iodine complexes, cadexomer iodine, polyvinylpyrrolidone iodine (PVP-I), sodium periodate, oxygen-generating agents including urea hydrogen peroxide and calcium peroxide, honey and other therapeutic materials known to the art can be added to the fibrous web. In an embodiment, the therapeutic materials are provided in the form of nanoparticles distributed homogenously throughout the superabsorbent core.

In still another embodiment, the fibers are spun from water-soluble polymers including polyacrylamides, polyacrylates acrylamide dimethylaminoethyl acrylate copolymers, polyamines, polyethyleneimines, polyamidoamines and polyethylene oxides including poly 2-ethyl-2-oxazoline).

In yet another embodiment, water-soluble fibers are impregnated with functional materials including growth factor-containing and hemostatic materials to be released in a controlled manner. Growth factor-containing materials to be used in the described embodiment include Epidermal Growth Factors (EGF), Transforming Growth Factors (TGF), Vascular Endothelial Growth Factors (VEGF), Fibroblast Growth Factors (FGF), Platelet-Derived Growth Factors (PDGF), Interleukins, Colony-Stimulating Factors (CSF) and Keratinocyte growth factors. In an embodiment, freeze-dried platelets are the source of growth factors. The soluble fibers dissolve upon contact with a fluid to dispense the functional materials at a controlled rate.

### Wetting Agents

Fibrillated polymeric fine fibers, processed in accordance with the present disclosure, can produce an absorbent material with unique wettability. Polypropylene (PP) fibers have grown to be one of the dominant materials in the nonwovens industry. It is estimated that over 90% of all Melt-blown (MB) nonwovens are made from Polypropylene (PP), because of its low cost, ease of processing, favorable chemical and physical properties, such as lack of heat shrinkage, impact strength, tensile strength, and its ability to be drawn into very fine fibers and nanofibers. However, PP is a typical hydrophobic polymer, so its melt-blown nonwovens have poor hydrophilicity, which limits their use in some areas. To improve wettability and increase the surface energy of PP nonwoven fabrics, many techniques have been studied to introduce polar groups to the surface and enrich surface functionality. Surface coatings with a solution containing hydrophilic substances such as surfactants and materials added to the melt that exhibit controlled migration to the surface of the PP nonwoven fabrics have also been used to improve the hydrophilic properties of polymers such as PP.

In an embodiment, wettability or hydrophilicity of the material is imparted through the addition of a wetting agent. In an embodiment of the disclosure the wetting agent is a nonionic surfactant. In an embodiment of the disclosure, a neat nonionic liquid surfactant is injected into the heated pressurized gas stream at about 2% by weight of the polymer before supplying the stream to the nozzle where it is atomized. The surfactant droplets are then mixed with the polymer stream in the nozzle, coating the polymer particles before fibrillation. Preferably, the nonionic surfactant is a “green” surfactant, not petroleum based but produced from renewable resources as well as biocompatible. In an embodiment of the disclosure, nonionic surfactants include products with good wettability characteristic, typically with an HLB (hydrophile-lipophile balance) value preferably between about 7 and about 13, Surfactants that can be used in the compositions include products based upon derivatized alkyl polyglucosides including cocoglucosides hydroxpropyl phosphate such as Suga®Fax D-86 (Colonial Chemical Inc.), organosilicone surfactants including trisiloxane ethoxylates such as Silwet™ L-77 (Momentive Performance Materials Inc.), alkylphenol ethoxylates including octylphenol ethoxylates such as Triton™ X-100 (Dow Chemical Inc.) and nonylphenol ethoxylates. It will be understood that surfactants other than those listed above may be used to improve wettability of the superabsorbent materials. A range of wetting agents may be employed and may be selected from the group consisting of ethoxylated nonyl phenol, sodium stearate, sodium dodecyl sulfate, sodium dodecylbenzene sulfonate, lauralamine hydrochloride, trimethyl dodecylammonium chloride, cetyl trimethylammonium chloride, polyoxyethylene alcohol, alkyphenolethoxylate, Polysorbate 80, propylene oxide modified polymethylsiloxane, dodecyl betaine, lauramidopropyl betaine, cocoamido-2-hydroxy-propyl sulfobetaine, alkyl aryl sulfonate, fluorosurfactants and perfluoropolymers and terpolymers.

### Ion Exchange Polymers

Ion-exchange polymers are widely used in different separation, purification, and decontamination processes. The most common examples are water softening and water desalinization. They have also been used in absorbent materials for fluids containing electrolytes to overcome the salt-poisoning effect that inhibits the absorbency of many SAP materials. Water-absorption and water-retention characteristics of SAPS are attributed to the presence of ionizable functional groups in the polymer structure. The ionizable groups typically are carboxyl groups, a high proportion of which are in the salt form when the polymer is dry, and which undergo dissociation and salvation upon contact with water. In the dissociated state, the polymer chain contains a plurality of functional groups having the same electric charge and, thus, repel one another. This electronic repulsion leads to expansion of the polymer structure, which, in turn, permits further absorption of water molecules. Without bound by theory, it is claimed that the presence of a significant concentration of electrolytes interferes with dissociation of the ionizable functional groups, and leads to the “salt poisoning” effect. Dissolved ions, such as sodium and chloride ions, therefore, have two effects on SAP particle. The ions screen the polymer charges and the ions eliminate the osmotic imbalance due to the presence of counter ions inside and outside of the particle. The dissolved ions, therefore, effectively convert an ionic particle into a nonionic particle, and swelling properties are lost. The removal of ions from electrolyte-containing solutions is often accomplished using ion exchange polymers. In this process, deionization is performed by contacting an electrolyte-containing solution with two different types of ion exchange polymers, i.e., an anion exchange polymer and a cation exchange polymer. The most common deionization procedure uses an acid polymer (i.e., cation exchange) and a base polymer (i.e., anion exchange). WO 96/17681 discloses admixing discrete anionic SAP particles, such as polyacrylic acid, with discrete polysaccharide-based cationic SAP particles to overcome the salt poisoning effect. Similarly, WO 96/15163 discloses combining a cationic SAP having at least 20% of the functional groups in a basic (i.e., OH) form with a cationic exchange resin, i.e., a nonswelling ion exchange resin, having at least 50% of the functional groups in the acid form.

In accordance with the principles of the present disclosure, it has been found that a superabsorbent nonwoven fibrous material having a combination of cationic and anionic SAP particles that are essentially unneutralized (0% to about 25% neutralized) can be manufactured in a single step using the coform process of the copending U.S. patent application Ser. No. 14/148,712 containing 50%-80% by weight of the combination of anionic and cationic SAP particles, added to the polymeric fibers form the superabsorbent material articles of the present disclosure. The acidic water-absorbing polymer typically is a lightly crosslinked acrylic-type polymer, such as lightly crosslinked polyacrylic acid or starch-graft polyacrylonitrile. The basic water-absorbing polymer typically is a lightly crosslinked acrylic type polymer, such as a poly(vinylamine) or a poly(dialkylaminoalkyl(meth)acrylamide). The basic polymer also can be a polymer such as a lightly crosslinked polyethylenimine, a poly(allylamine), a poly(allylguanidine), a poly(dimethyldi-allylammonium hydroxide) or a guanidine-modified polystyrene.

The superabsorbent materials of the present disclosure exhibit exceptional water absorption and retention properties, especially with respect to electrolyte-containing liquids such as saline, blood, urine, and menses even when containing 20-50% fiber, with or without a synthetic binder. In addition, the sheet materials have an ability to absorb liquids quickly, demonstrate good wettability and conductivity into and through the SAP particles, and have a high loft and structural integrity such that the superabsorbent material, upon hydration, resists deformation under an applied stress or pressure, when used alone or in a multi-layered superabsorbent article.

### Multi-Layered Superabsorbent Articles

In another embodiment, the present disclosure is directed to multi-layered superabsorbent articles of improved absorbency characteristics. Generally, absorbent articles are designed to have at least four distinct layers:
(1) a thin hydrophilic topsheet of bonded fibers in contact with the user's skin usually referred to as the contact layer;
(2) a lofty hydrophilic non-woven layer referred to as the distribution layer containing hydrophilic fibers to rapidly absorb the liquid from the contact layer and pass it to the superabsorbent core layer.
(3) a superabsorbent core containing mainly hydrophilic fibers and SAP particles often contributing as much as 90% of the overall thickness of the article; and
(4) a liquid barrier outer layer referred to as the backing layer which is usually a thin non-woven layer made from hydrophobic polyolefin fibers.

### Contact Layer

The first layer of the superabsorbent material is the layer in contact with the skin. This is called a top sheet or contact layer. In an embodiment of the disclosure, the contact layer is made of hydrophilic nonwoven materials. N I another embodiment, the surface of the contact layer is modified to be hydrophilic to enable fast absorption of all liquid that is in contact with the wearer's skin. In another embodiment, the contact layer is inherently non-adherent to the skin surface or modified to be non-adherent using various possible methods including those known to the art. In an example of an embodiment of the disclosure, Reon™-iodine complex powder is coated on the contact layer fibers forming a non-adherent hydrogel upon contact with body fluids and, in turn, forms a non-adherent coating around the fibers.

Fiber components of the contact layer are formed from any polymer known to be usable in the art of fiber spinning including biocompatible polymers comprising poly(esters) based on polylactide (PLA), polyglycolide (PGA), polycaprolactone (PCL), and their copolymers or modified poly(saccharide)s, e.g., starch, cellulose, and chitosan. Fiber components of the contact layer are spun in a way to give the fibers the ability to compress as with the process described in U.S. Pat. No. 8,668,854 to (Marshall et al.). Specifically, the compressibility of the fiber matrix is a result of having a high loft and porosity. The high loft is a result of the fiber matrix being comprised of a first distribution of polymeric nanofibers and a second distribution of polymeric fine fibers homogeneously dispersed throughout the contact layer. The porosity, or the volume of fiber pores divided by the total fiber volume, is preferably greater than 85 percent and more preferably greater than 90 percent. When the superabsorbent layer expands upon taking in liquid, the fibers compress to keep the article from applying pressure to the outside surface of the contact layer due to the expansion. Additionally, the porosity of the fiber matrix decreases upon compression to further restrict the liquid from leaving the superabsorbent layer and exiting the article through the external surface of the contact layer.

In an embodiment of the disclosure, the contact layer is biocompatible, ie. nonhemolytic and nonpyrogenic as well as noncytotoxic.

In another embodiment, the contact layer can be engineered to change color to indicate the need for change or removal fo the multilayered material. In an example, fibers impregnated with Reon™-iodine hydrogel complex powder have a dark purple color typical to a starch-iodine complex that fades as the iodine is released. Other compounds, such as calcium sulfate, that change color upon exposure to moisture, such as wound fluid, can be coated on the fibers. To observe the color of the contact layer without disturbing the dressing, a window can be created in the contact layer surface.

### Distribution Layer

Optionally, the multilayered material may include a second layer between the contact layer and the superabsorbent layer. This second layer is called a distribution layer. This layer is what drains the contact layer of moisture. The distribution layer also disperses all the liquid from the contact layer sheet as much as possible, to increase the contact area of the absorption core and to keep the core from getting locally saturated. The distribution layer is made of hydrophilic fibers. In an embodiment of the disclosure, the surface of the fibers is treated with a surfactant to impart hydrophilic properties to the material. In another embodiment, of the disclosure a hydrophilic compound comprising a surfactant is added to the polymer melt. When the product is subjected to external pressure between the top sheet and the absorption core, the distribution material works as a protection. A distribution layer with good mechanical properties prevents liquid from easily traveling through.

In an embodiment of the disclosure, the distribution layer comprises a hydrophilic three-dimensional fibrous web consisting of a first population of fibrillated nanofibers, and a second population of fibrillated microfibers, both populations uniformly distributed throughout the three-dimensional fibrous web where the first population comprises at least 50% of the total fiber population.

### Backing Layer

Backing layers which are permeable to vapor are known as breathable layers and have been described in the art. These breathable backing layers provide a cooler garment and permit some drying of the superabsorbent material while it is being used. In general, these breathable backing layers are intended to allow the passage of vapor through them while retarding the passage of liquid to the outside or bacteria from the outside.

An overly breathable backing layer may result in excessive amount of vapor transmitted through the backing layer as well as dryness and sticking of the material to the skin. This may also increase the amount of unpleasant odors as more volatile compounds are transmitted through the backing sheet together with water vapor. Conversely, an excessively vapor resistant material may no longer be able absorb additional fluid and may feel uncomfortably wet against the skin. In an embodiment of the disclosure, the backing layer is tailored to have a moisture vapor transmission rate (MVTR) which is a function on the amount of liquid absorbed in order to inhibit excessive amount of moisture from being transmitted through the backing sheet. Such a capability is possible by using a backing layer material through which MVTR increases with increased fluid pressure. Therefore, if more fluid is absorbed and in turn, puts pressure on the backing layer, the MVTR increases and more vapor is allowed to pass through.

In another embodiment, the backing layer has an MVTR of 800 g/m2/24 hr to 2,000 g/m2/24 hr.

In yet another embodiment, the backing layer has a hydrostatic head greater than 30 cm.

### Coform Process Description

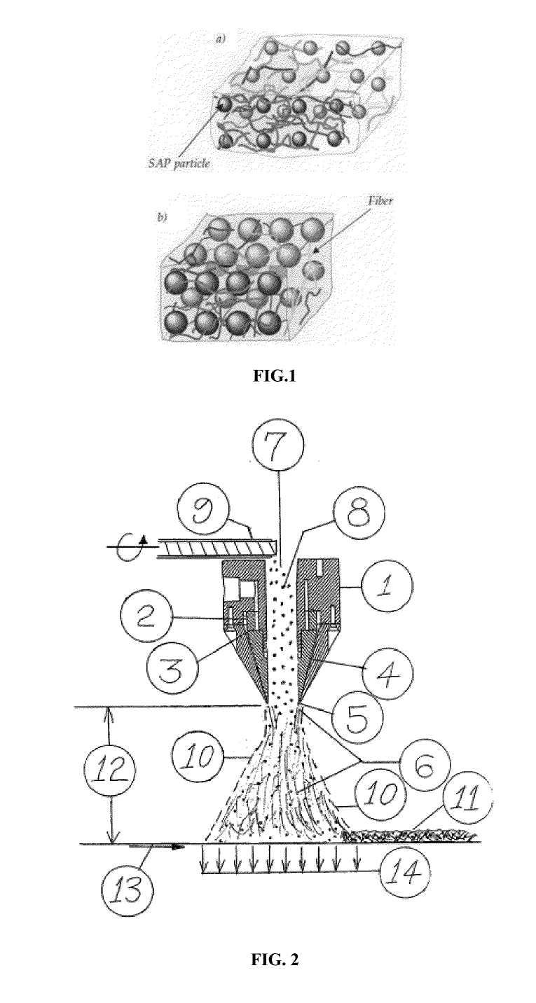

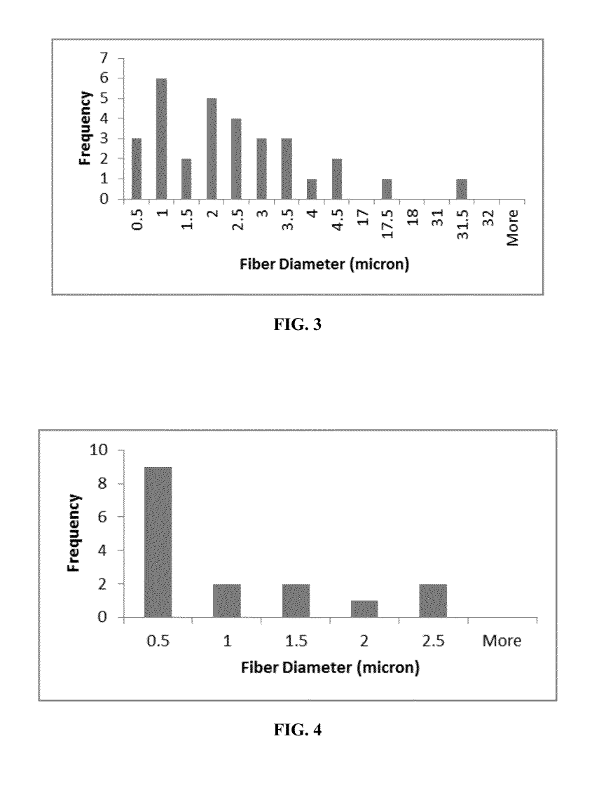

In an embodiment, the superabsorbent material is produced according to the coforming process detailed in copending U.S. patent application Ser. No. 14/148,712. FIG. 2 illustrates an embodiment of the coforming process. The fiber spinning nozzle 1 shown in cross-section in FIG. 2 is of an axisymmetric design. Heated gas is injected into a swirl chamber 2 by two orifices, creating a swirling rotating flow about the axis of the nozzle. In an embodiment, a surfactant is injected into the heated gas stream, prior to the combined flow of surfactant and heated gas being injected into the nozzle swirl chamber 2. A heated polymer melt, comprising a mixture of substances is injected into the swirl chamber 2 through orifices 3. The swirling, rotating gas flow deforms and mixes with the polymer (mixture of substances) forming a two-phase (gas-polymer) flow. The two-phase flow traverses a narrow flow channel 4 forming a polymer film at the exit gap 5. At the exit gap the polymer film is broken into discrete elements or streams which are attenuated to become polymeric fibers 6. The axisymmetric nozzle 1 contains a hollow cylindrical hole 7. The hot gas jet issuing from axisymmetric gap 5 creates a negative pressure in this region which aspirates gas through the hollow cylinder 7. The gas flow naturally aspirated through hollow cylinder 7 enables powder particles 8 from feed apparatus, here a screw 9 to be aspirated directly into the fiber making process. The powder particles 8 are substantially completely enveloped and contained within the fiber making stream. They are both thermally bonded onto the fibers and entrapped within the fibrous structure of the envelope of the forming jet 10, such that very few powder particles escape. The powder particles are efficiently contained in the web 11. The nozzle gap 5 is located at a distance 12 from a collecting surface 13. The fibers with attached powder are formed into a sheet or web material 11 by vacuum 14 and a moving collection surface 13.

### EXAMPLES

The examples provided below show the incorporation of fine, ultrafine, or nanosize particles into a non-woven fibrous structure. Examples include various superabsorbent particles, powdered activated carbon, copper oxide particles (0.5 micron), and atomized droplets of a nonionic surfactant. In each case, the fibrous structure is coformed with the particulates, making it practical to manufacture the new material by coform melt-film fibrillation processes such as described in copending U.S. patent application Ser. No. 14/148,712. In several examples, the fibrous structure is formed into a homogenous monolitic layer. In another example, the fibrous structure is formed into more than one layer. In yet another example, the fibrous structure is calendered.

### Example 1

### Jet Milled Sodium Polyacrylate

EXAMPLE 1

| Polymer | Polypropylene (PP) |
|---|---|
| SAP | Sodium Polyacrylate (SPA) |
| Median Nanofiber size | 0.8 microns |
| Median Microfiber Size | 3 microns |
| Particle Size | <30 microns |
| Material basis weight | 48 gsm |
| Particle weight % | 38% |

An extruder (¾ inch Laboratory Extruder from C. W. Brabender) was used to supply a polymer mixture to a spin nozzle illustrated in FIG. 2. The polymer mixture was 45% by weight isotatic polypropylene with molecular weight 12,000, 45% by weight isotatic polypropylene with molecular weight 30,000, and 10% by weight atatic polypropylene with molecular weight 14,000. The nozzle exit gap 5 was 0.51 mm. The diameter of the hollow aspiration cylinder 7 was 25.4 mm. The polymer temperature at the extruder exit was 200 C and the polymer pressure at the extruder exit was 8.6 bars. The polymer mixture was injected into nozzle 1 through eight orifices 3 each with diameter=0.51 mm. Heated air was injected into swirl chamber 2 at 265 C. The air flowrate was 0.16 cubic m per minute at 4.1 bars. Nozzle 1 produced nanofibers with median fiber size=0.8 microns and microfibers with median fiber size=3 microns, (See FIG. 3). The fiber flowrate was 4.0 g per minute. A SAP polymer powder that had been jet milled to particles sizes of about 30 microns and smaller was aspirated into the cylinder 7. The super absorbent powder was sodium polyacrylate (SPA) Water-Loc GB-6B from Educational Innovations Inc. A collecting surface 13 was located approximately 22.8 cm from nozzle exit 5. The nanofibers and micro fibers produced at the nozzle exit 5 and the entangled SAP powder particles where collected on surface 13. The collected material had a basis weight of approximately 48 gsm with the jet milled powder weighing about 18 gsm.

### Example 2

### Reon™ SAP Particles

EXAMPLE 2

| | |
|---|---|
| Polymer | Polypropylene (PP) |
| SAP | Reon ™ |
| Median Nanofiber size | 0.34 microns |
| Median Microfiber Size | 3 microns |
| Median Particle Size | 75 microns |
| Material basis weight | 250 gsm |

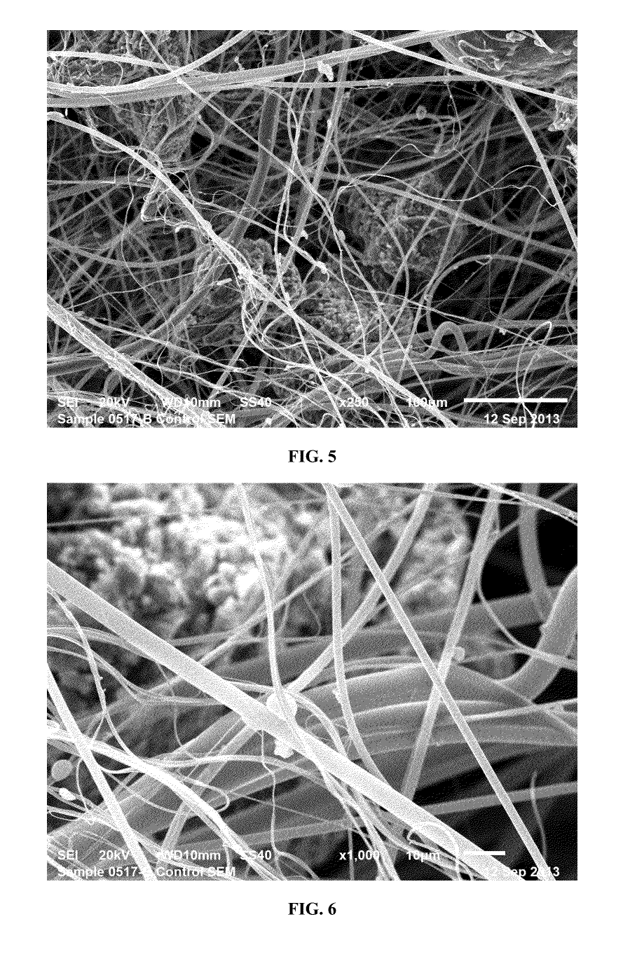

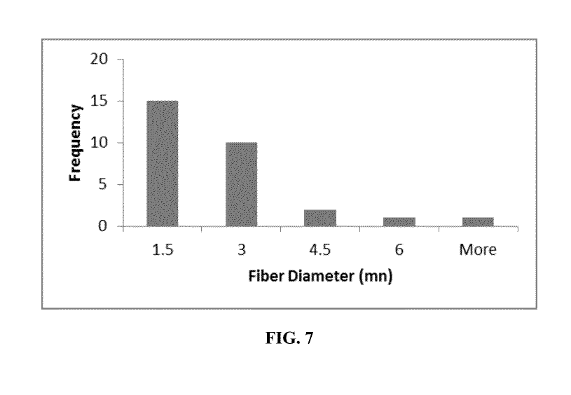

An extruder (¾ inch Laboratory Extruder from C. W. Brabender) was used to supply a polymer mixture to a spin nozzle having configuration illustrated in FIG. 2. The polymer mixture was 95% by weight isotatic polypropylene with molecular weight 12,000 and 5% by weight Techsurf hydrophilic polymer masterbatch PPM 15560. The nozzle exit gap 5 was 0.38 mm. The diameter of the hollow aspiration cylinder 7 was 25.4 mm. The polymer temperature at the extruder exit was 197 C and the polymer pressure at the extruder exit was 3.8 bars. The polymer mixture was injected into nozzle 1 through 8 orifices 3. Heated air was injected into swirl chamber 2 at 265 C. The air flowrate was 0.18 cubic m per minute at 2.8 bars. Nozzle 1 produced nano and micro fibers with median fiber size=0.34 micron, average fiber size=0.76 micron, and standard deviation=0.76 microns (the fiber size distribution is shown in FIG. 7.) A superabsorbent polymer powder Reon™, a saponified starch-graft polyacrylonitrile copolymer made from corn, with particle size range=74-420 microns was aspirated into the cylinder 7. A collecting surface 13 was located approximately 43.2 cm from nozzle exit 5. The nanofibers produced at the nozzle exit 5 and the entangled powder particles of Reon™ where collected on surface 13. SEM pictures of the collected fibers and Reon™ particles are shown in FIG. 5 and FIG. 6. The collected material had a basis weight of approximately 250 gsm. The collected material was sprayed with a mixture of liquid organosilicone surfactant (Silwet L-77 made by Momentive) at 10% by weight and a mixture of MeOH/water at 90% by weight. The weight ratio of the MeOH/water mixture was 95% by weight MeOH and 5% by weight water. After drying in ambient temperature air, the saline solution absorbance capacity was found to about 31 g saline/g material and the rate of saline solution absorbance was found to be about 0.9 10 ml saline/g material/sec.

### Example 3

### SAP AP-75 with Sprayed Surfactant

EXAMPLE 3

| | |
|---|---|
| Polymer | Polypropylene (PP) |
| SAP | AP-75 |
| Median Nanofiber size | 0.8 microns |
| Median Microfiber Size | 3 microns |
| Median Particle Size | 150 microns |
| Material basis weight | 272 gsm |

An extruder (¾ inch Laboratory Extruder from C. W. Brabender) was used to supply a polymer mixture to a spin nozzle illustrated in FIG. 2. The polymer mixture was 45% by weight isotatic polypropylene with molecular weight 12,000, 45% by weight isotatic polypropylene with molecular weight 30,000, and 10% by weight atatic polypropylene with molecular weight 14,000. The nozzle exit gap 5 was 0.51 mm. The diameter of the hollow aspiration cylinder 7 was 25.4 mm. The polymer temperature at the extruder exit was 186 C and the polymer pressure at the extruder exit was 5.6 bars. The polymer mixture was injected into nozzle 1 through 8 orifices 3 each with diameter=0.51 mm. Heated air was injected into swirl chamber 2 at 265 C. The air flowrate was 0.16 cubic m per minute at 4.1 bars. Nozzle 1 produced nano and micro fibers with median fiber size=1.9 microns, average fiber size=3.36 micron, and standard deviation=5.94 microns (the fiber size distribution is shown in FIG. 3.) The fiber flowrate was 4.6 g per minute. A superabsorbent polymer powder (AP-75 from Evonik) with particle size range=150-850 microns was aspirated into the cylinder 7 at a rate of approximately 15 g per minute. A collecting surface 13 was located approximately 43.2 cm from nozzle exit 5. The nano and micro fibers produced at the nozzle exit 5 and the entangled superabsorbent powder particles where collected on surface 13. The collected material had a basis weight of approximately 272 gsm. The collected material was sprayed with a mixture of liquid organosilicone surfactant (Silwet L-77 made by Momentive) at 10% by weight and a mixture of MeOH/water at 90% by weight. The weight ratio of the MeOH/water mixture was 95% by weight MeOH and 5% by weight water. After drying in ambient temperature air, the saline solution absorbance capacity was found to about 19 g saline/g material and the rate of saline solution absorbance was found to be about 0.6 10 ml saline/g material/sec.

### Example 4

### Jet Milled Polyacrylamide with Atomized Surfactant

EXAMPLE 4

| | |
|---|---|
| Polymer | Polypropylene (PP) |
| Particle | Sodium Polyacrylate (SPA GB-6B) |
| Surfactant | Organosilicone (Silwet L-77) |
| Median Nanofiber size | 0.8 microns |
| Median Microfiber Size | 3 microns |
| Median Particle Size | <40 microns |
| Material basis weight | 234 gsm |
| Absorbent capacity | 19 (g saline/g material) |
| Absorbency rate | 1.3 (10 ml/g material/s) |

An extruder (¾ inch Laboratory Extruder from C. W. Brabender) was used to supply a polymer mixture to a spin nozzle illustrated in FIG. 2. The polymer mixture was 45% by weight isotatic polypropylene with molecular weight 12,000, 45% by weight isotatic polypropylene with molecular weight 30,000, and 10% by weight atatic polypropylene with molecular weight 14,000. The nozzle exit gap 5 was 0.51 mm. The diameter of the hollow aspiration cylinder 7 was 25.4 mm. The polymer temperature at the extruder exit was 188 C and the polymer pressure at the extruder exit was 6.2 bars. The polymer mixture was injected into nozzle 1 through 8 orifices 3. Heated air was injected into swirl chamber 2 at 268 C. The air flowrate was 0.16 cubic m per minute at 4.1 bars. Nozzle 1 produced nano and micro fibers with average fiber size=3.36 micron, median fiber size=1.9 microns, and standard deviation=5.94 microns (the fiber size distribution is shown in FIG. 3) The fiber flowrate was 7.2 g per minute. A superabsorbent polymer powder was made from polyacrylamide particles (from Pfaltz & Bauer) by jet mill grinding the particles into a powder with particle size range <40 microns microns. The jet milled powder was aspirated into the cylinder 7 at a rate of approximately 4 g per minute. Liquid organosilicone surfactant (Silwet L-77 made by Momentive) was injected into the heated air stream prior to the combined flow of surfactant and heated air being injected into the nozzle swirl chamber 2. The liquid surfactant was vaporized as it mixed with the heated air in the swirl chamber 2. A collecting surface 13 was located approximately 43.2 cm from nozzle exit 5. The nano and micro fibers produced at the nozzle exit 5 and the entangled superabsorbent powder particles where collected on surface 13. The collected material had a basis weight of approximately 234 gsm. The saline solution absorbance capacity was found to be about 19 g saline/g material and the rate of saline solution absorbance was found to be about 1.3 10 ml saline/g material/sec.

### Example 5

### Sodium Polyacrylate with Atomized Surfactant

EXAMPLE 5

| | |
|---|---|
| Polymer | Polypropylene (PP) |
| Particle | Sodium Polyacrylate (SPA GB-6B) |
| Surfactant | Alkyl Polyglocoside (Suga ® Fax D86) |
| Median Nanofiber size | 0.5 microns |
| Median Microfiber Size | 2 microns |
| Median Particle Size | <40 microns |
| Material basis weight | 175 gsm |
| Material thickness | 0.7 mm |
| Absorbent capacity | 32 (g saline/g material) |
| Absorbency rate | 2.3 (10 ml/g material/s) |

An extruder (¾ inch Laboratory Extruder, C. W. Brabender) was used to supply a polymer mixture to a spin nozzle as illustrated in FIG. 2. The polymer mixture was 40% by weight isotatic polypropylene with molecular weight 12,000, 40% by weight isotatic polypropylene with molecular weight 30,000, and 20% by weight atatic polypropylene with molecular weight 14,000. The nozzle exit gap 5 was 0.36 mm. The diameter of the hollow aspiration cylinder 7 was 19.1 mm. The polymer temperature at the extruder exit was 170 C and the polymer pressure at the extruder exit was 15.8 bars. The polymer mixture was injected into nozzle 1 through 6 orifices 3. Liquid sodium cocoglucosides hydroxpropyl phosphate (Suga®Fax D86) was injected at 0.39 g per minute into an air stream heated to 265 C, prior to the combined flow of surfactant and heated air being injected into the nozzle swirl chamber 2. The liquid surfactant was atomized as it mixed with the heated air in the swirl chamber 2. The air flowrate was 0.14 cubic m per minute at 4.1 bars. Nozzle 1 produced nanofibers with median fiber size=0.5 microns and microfibers with median fiber size=2 microns. The fiber flowrate was 3.9 g per minute. Sodium polyacrylate powder (GB-6B Water-Lock®) was aspirated into the cylinder 7. A collecting surface 13 was located approximately 25.4 cm from nozzle exit 5. The nanofibers and microfibers produced at the nozzle exit 5 and the entangled sodium polyacrylate powder particles where collected on surface 13 and where found to be hydrophilic. The collected material had a basis weight of approximately 175 gsm. The collected material was calendared in a Carver press at about 10,000 psi for about 15 sec, resulting in a material thickness of about 0.7 mm. The saline solution absorbent capacity was about 32 g saline/g material and the absorbency rate was about 2.3 (10 ml saline/g material/sec).

### Example 6

### SAP+Copper Oxide Nanoparticles

EXAMPLE 4

| | |
|---|---|
| Polymer | Polypropylene (PP) |
| Median SAP Particle size | 150 microns |
| Nanoparticle | Copper Oxide |
| Median Nanofiber size | 0.5 microns |
| Median Microfiber Size | 2 microns |
| Median Nanoparticle Size | 0.5 microns |
| Material basis weight | 50.2 gsm |

An extruder (¾ inch Laboratory Extruder from C. W. Brabender) was used to supply a polymer mixture to a spin nozzle illustrated in FIG. 2. The polymer mixture was 45% by weight isotatic polypropylene with molecular weight 12,000, 45% by weight isotatic polypropylene with molecular weight 30,000, and 10% by weight atatic polypropylene with molecular weight 14,000. The nozzle exit gap 5 was 0.51 mm. The diameter of the hollow aspiration cylinder 7 was 25.4 mm. The polymer temperature at the extruder exit was 193 C and the polymer pressure at the extruder exit was 8.1 bars. The polymer mixture was injected into nozzle 1 through 8 orifices 3 each with diameter=0.51 mm. Heated air was injected into swirl chamber 2 at 265 C. The air flowrate was 0.16 cubic m per minute at 4.1 bars. Nozzle 1 produced nano and micro fibers with median fiber size=1.9 microns, average fiber size=3.36 micron, and standard deviation=5.94 microns (the fiber size distribution is shown in FIG. 23) Copper oxide powder with median particle size 0.5 micron was pre-mixed in a container with superabsorbent polymer powder (AP-75 from Evonik) with particle size range=150-850 microns. The weight ratio was: 0.5% copper oxide powder and 99.5% SAP powder. The smaller copper oxide particles were attached to the larger SAP particles. The combined copper oxide and SAP particles were aspirated into the cylinder 7. A collecting surface 13 was located approximately 43.2 cm from nozzle exit 5. The nano and micro fibers produced at the nozzle exit 5 with attached (and entangled) combined copper oxide and SAP particles where collected on surface 13.

What is claimed is:

1. A coform superabsorbent core material comprising
a) a hydrophilic three-dimensional fibrous web comprising a first population of fibrillated nanofibers and a second population of fibrillated microfibers, both populations uniformly distributed throughout the three-dimensional fibrous web wherein the first population comprises at least 50% of the total fiber population;
b) a population of superabsorbent polymer (SAP) particles with a median size of less than 40 microns dispersed throughout the fibrous web.

2. The coform superabsorbent core material of claim 1 having a saline solution absorbent capacity greater than about 30 times its dry weight and the rate of absorbency is greater than about 20 ml of saline solution per gram of material per second.

3. The coform superabsorbent core material of claim 1 wherein the polymeric fine fibers composition comprises at least one polymer selected from the list consisting of (polypropylene (PP), polybutylene terephthalate (PBT), polybutylene succinate (PBS), polyethylene (PE), polyethylene terephthalate (PET), polystyrene (PS), polyacrylonitrile (PAN), polycarbonate (PC), polyvinylidene fluoride (PVDF), polyvinylidene chloride (PVDC), polyphenylene sulfide (PPS), polytetrafluoroethylene (PTFE), polyethersulfone (PES), polysulfone (PSU), polymethyl methacrylate (PMMA), polyurethane (PUR), polyamide (PA), polyvinyl chloride (PVC), polylactic acid (PLA), polyglycolic acid (PGA), polylactic-co-glycolic acid (PLGA), polycaprolactone (PCL)).

4. The coform superabsorbent core material of claim 1 where the SAP is selected from the list consisting of cross-linked polyacrylates and polyacrylamides, cross-linked copolymers of maleic anhydride, polyvinyl alcohol, polyvinyl ethers, hydroxypropyl-cellulose (HPC), carboxymethyl-cellulose (CMC), carboxymethyl starch (CMS), polymers and copolymers of vinyl sulfonic acid, graft copolymers on polysaccharides such as chitin, chitosan, cellulose, starch, natural gums and polypeptide-based copolymers including saponified starch-graft polyacrylonitrile copolymer made from corn (Reon™).

5. The coform superabsorbent core material of claim 1 wherein the porosity is greater than 75%.

6. The coform superabsorbent core material of claim 1 wherein the SAP loading capacity is greater than 60% of the total weight of the superabsorbent material.

7. The coform superabsorbent core material of claim 1 further comprising a wetting agent selected from the group consisting of ethoxylated nonyl phenol, sodium stearate, sodium dodecyl sulfate, sodium dodecylbenzene sulfonate, lauralamine hydrochloride, trimethyl dodecylammonium chloride, cetyl trimethylammonium chloride, polyoxyethylene alcohol, alkyphenolethoxylate, Polysorbate 80, propylene oxide modified polymethylsiloxane, dodecyl betaine, lauramidopropyl betaine, cocoamido-2-hydroxy-propyl sulfobetaine, alkyl aryl sulfonate, fluorosurfactants and perfluoropolymers and terpolymers.

8. The coform material of claim 1 comprising a nonionic surfactant selected from the group consisting of derivatized alkyl polyglucosides, trsiloxane ethoxylates, octylphenol ethoxylates and nonylphenol ethoxylates.

9. The coform superabsorbent core material of claim 1 comprising odor controlling nanoparticles selected from the group consisting of activated carbon, clays, silicas, zeolites, and molecular sieves.

10. The coform superabsorbent core material of claim 1 comprising metal ion nanoparticles selected from the group consisting of gold, silver, platinum, palladium, copper, tin, cobalt, manganese, bismuth, lead and zinc.

11. The coform material of claim 1 further comprising urease inhibitors selected from the list consisting of N-(n-butyl)thiophosphoric triamide, N-(n-butyl)phosphoric triamide, thiophoshoryl triamide, phenyl phosphorodiamidate, cyclohexyl thiophosphoric triamide, cyclohexyl phosphoric triamide, phosphoric triamide, hydroquinone, P-benzoquinone, hexaamidocyclotriphosphazene, thiophyridines, thiophyrimidines, thiophyridine-Noxides, NN-dihalo-2-imidazolidinone, N-halo-2-oxazolidinone

12. The coform superabsorbent core material of claim 1 further comprising therapeutic agents selected from the list consisting of iodine delivery agents, ion exchange agents, oxygen delivery agents and honey.

13. The coform superabsorbent core material of claim 1 further comprising a hydrophilic contact layer of bonded fibers.

14. The coform superabsorbent material of claim 1 further comprising a non-woven distribution layer comprising hydrophilic fibrillated fine fibers engineered to rapidly absorb a liquid and pass it to the superabsorbent core layer.

15. The material of claim 14 wherein the density, in grams per cubic centimeter is less than 0.25.

16. The coform superabsorbent material of claim 1 further comprising a liquid impermeable and vapor permeable backing layer comprising hydrophobic fine fibers.

17. The material of claim 16 wherein the backing layer has an MVTR between 800 and 2000 and a hydrostatic head greater than 30 cm.

18. The coform material of claim 1 further comprising fibers spun from water-soluble polymers selected from the list consisting of polyacrylamides, polyacrylates acrylamide dimethylaminoethyl acrylate copolymers, polyamines, polyethyleneimines, polyamidoamines and polyethylene oxides including poly 2-ethyl-2-oxazoline.

19. The material of claim 18 wherein the water soluble fibers are impregnated with growth factor-containing materials selected from the list consisting of Epidermal Growth Factors (EGF), Transforming Growth Factors (TGF), Vascular Endothelial Growth Factors (VEGF), Fibroblast Growth Factors (FGF), Platelet-Derived Growth Factors (PDGF), Interleukins, Colony-Stimulating Factors (CSF) and Keratinocyte growth factors.

20. The coform superabsorbent core material of claim 1 wherein the population of SAP particles comprises a first distribution of anionic SAP particles selected from the list consisting of lightly crosslinked polyacrylic acid, starch-graft polyacrylonitrile and a second distribution of cationic SAP particles selected from the list consisting of polyvinylamine, polydialkylaminoalkyl methacrylamide, lightly crosslinked polyethylenimine, a polyallylamine, a polyallylguanidine, a polydimethyldi-allylammonium hydroxide or a guanidine-modified polystyrene and wherein the median anionic and cationic SAP particle sizes are less than 40 microns and wherein less than 25% of the SAP particles are neutralized.
